(19) United States
(12) Patent Application Publication
(10) Pub. No.: US 2007/0010814 A1
(43) Pub. Date: Jan. 11, 2007

(54) Device for extending bones

(76) Inventor: Roman Stauch, Assarnstadt (DE)

Correspondence Address: BACHMAN & LAPOINTE, P.C., 900 CHAPEL STREET, SUITE 1201, NEW HAVEN, CT, 06510, US

(21) Appl. No.: 10/569,519

(22) PCT Filed: Jul. 22, 2004
(86) PCT No.: PCT/EP04/08194

§ 371 Date: Feb. 23, 2006

(30) Foreign Application Priority Data
Aug. 28, 2003 (DE) 10340025.7

(51) Int. Cl. A61F 2/30

(52) U.S. Cl. 606/62

(57) ABSTRACT

A device for extending bones, comprising two elements that can be displaced in relation to one another and that are interconnected by at least one drive element. When the two elements are displaced axially in relation to one another, they are guided in a manner that prevents relative radial torsion.

### BACKGROUND OF THE INVENTION

The present invention relates to a device for extending bones, with two elements that can be moved in relation to one another and that are interconnected via at least one drive element.

Devices of this kind are known and commonly available on the market in a wide variety of formats and designs. They are used in particular for distraction of bones. They can be inserted into a bone cavity or a medullary space of a bone, and distraction can take place after the bone has been cut through.

A disadvantage of conventional devices is that they do not have high distraction forces and are technically very complex and expensive to manufacture, and, in addition, are to be produced in a limited size. For this reason, the possible applications are very limited, particularly in bones of short length and small diameter.

An additional disadvantage is that conventional distraction devices have a short travel and, after the complete travel has been exhausted, other distraction devices or appliances have to be fitted, which is also undesirable.

WO 98/30163 A discloses a distraction device for moving apart two bone sections, where two slidably mounted cylindrical sleeves arranged coaxially one inside the other can be moved apart by means of a planetary roller system.

U.S. Pat. No. 5,601,551 discloses a distraction device in which a number of components are arranged such that they can be individually moved to and fro on a rigid bar, the distraction device engaging on the outside of a bone.

The object of the present invention is to make available a device of the type mentioned in the introduction which overcomes the stated disadvantages and with which a device is made available that permits high distraction forces and provides a very long travel.

In addition, a device of this kind is to be able to be produced in all possible sizes so that it can be fitted in any desired spaces. In addition, energy and data transmission, and activation, must be able to take place in an unproblematic and contactless manner.

### SUMMARY OF THE INVENTION

This object is achieved by the fact that, when the two elements are moved axially in relation to one another, they are guided in a manner secure against relative radial torsion, an inner cross section of the element in the area between an electric motor and planetary roller system has a polygonal, rectangular, many-cornered configuration as guide area for the second element, and an outer cross section of the element at least partially corresponds to the inner cross section of the first element in the guide area and has a polygonal, rectangular or many-cornered configuration.

In the present invention, it has proven particularly advantageous that the second element is fitted into the first element in such a way that it is secured against radial torsion relative to said first element. The securing against torsion can be obtained in different ways. For example, an outer contour or outer cross section of the second element can have a polygonal configuration, while a correspondingly configured guide element with correspondingly configured inner cross section of the second element in the end area ensures securing against radial torsion. This makes it possible, inside the second element, to ensure a distraction, in particular an axial movement of the second element in relation to the first element, by means of a planetary roller system or thread or spindle system or the like, by way of the drive element. This distraction, as a function of the gear ratio of the planetary roller system, ensures very high distraction forces, while at the same time an exact distance or distraction can also be traveled per unit of time. In addition, the distraction or each travel can be exactly activated and executed.

Moreover, corresponding force sensors fitted in the element and/or between a shaft and the drive element, in particular electric motor, can determine the distraction ratio on the basis of the forces applying as pressure forces or torques.

In this way, particularly in the distraction of bones, the distraction process can also be exactly controlled and monitored. In addition, a force-controlled distraction or axial movement of the two elements away from one another is also possible. This is likewise intended to lie within the scope of the present invention.

Moreover, the first element configured as receiving sleeve is assigned an electric motor, an electronics unit and, adjoining this, an energy and/or data transmission element. Radial locking bores are preferably provided in the end area of the two elements in order to fix the device, in particular the medullary nail configured as the device, in the bone, for example in a long bone, via fastening elements, screws, nails or the like (not shown here).

The device is preferably activated and powered in a contactless and inductive manner via the energy and/or data transmission elements.

### BRIEF DESCRIPTION OF THE DRAWINGS

Further advantages, features and particulars of the invention will become evident from the following description of preferred illustrative embodiments and from the drawing, in which:

FIG. 1 shows a schematic and partial longitudinal section through the first element in the end area;

FIG. 2a shows a schematic and partial longitudinal section through the first element according to FIG. 1;

FIG. 2b shows a schematic cross section through the second element according to FIG. 1;

FIG. 3 shows a schematic cross section through the elements 1 and 2 in a further illustrative embodiment.

### DETAILED DESCRIPTION

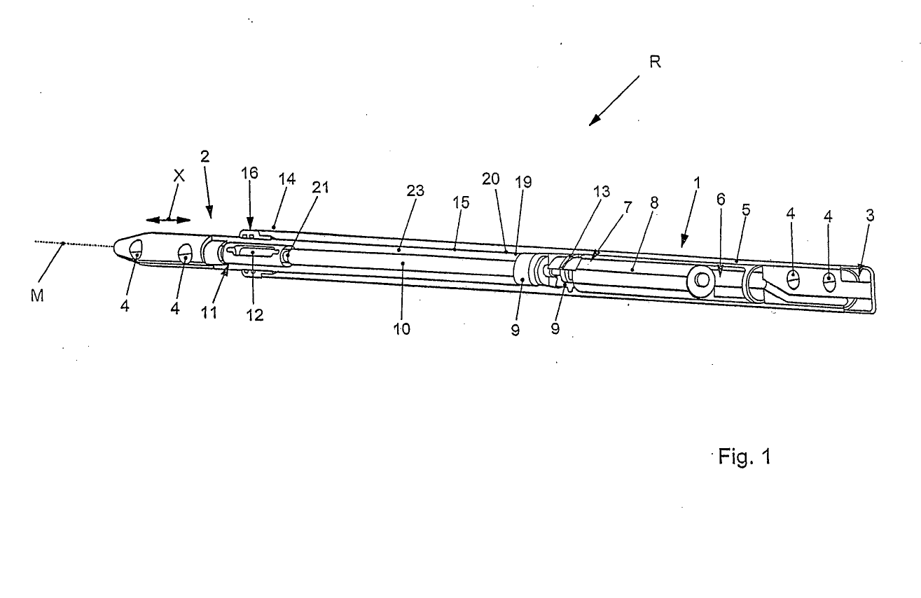

According to FIG. 1, a device R according to the invention comprises a first element 1, and a second element 2 that is guided linearly and axially in said first element 1.

Integrated in the first element 1, particularly in the end area, there is an energy and/or data transmission element 3 which delivers the required energy and ensures bidirectional and contactless exchange of data. In the end area 19 of the element 1, there are also at least two radial locking bores 4 which serve to fix the device R for example in a bone that is to be extended. At the same time the element 1 is secured against radial torsion relative to the bone during fixing.

The element 1 is preferably designed as a receiving sleeve 5 that comprises an electronics unit 6 (only symbolically indicated here) connected to the energy and/or data transmission element 3 and also to a drive element 7.

The drive element 7 comprises an electric motor 8 which sets a drive shaft 10 in a rotary movement via bearings 9 (only symbolically indicated here). Adjoining one end of the drive shaft 10 there is a planetary roller system 11 in which a plurality of planets (not shown in detail here) are provided which are driven via the drive shaft 10 and the planet carrier 12 (only symbolically indicated here).

The electric motor 8 is preferably adjoined by a force sensor 13 for determining the axial forces of the shaft and also the torques, which force sensor 13 is in turn connected to the electronics unit 6.

Between the electric motor 8 and an end area 14 of the first element 1, the latter has a guide area 15 in its inside, said guide area 15 preferably having a cylindrical configuration.

In the end area 14, a guide element 16 is fitted on the element 1, this guide element 16 having an inner cross section 17 that corresponds approximately to an outer cross section 18 of the second element 2.

Inner cross section 17 and outer cross section 18 are preferably of polygonal configuration in cross section. In this way it is possible to avoid radial torsion of the element 2 guided in the guide area 15 of the element 1. It is able to move axially to and fro along a center axis M, but it cannot twist radially.

The element 2 is preferably configured almost completely as a polygonal profile with regard to its outer cross section.

However, in its end area 19, its cross section can have another outer contour 20 approximately corresponding to the guide area 15 of the first element 1.

The inside of the element 2 is preferably configured as a thread which interacts with the planetary roller system 11, spindle system or the like or with its planets (not shown in detail here).

By means of suitable driving of the drive shaft 10 and of the planetary roller system 11, the element 2 can move out of the element 1 in the direction of the double arrow X, along a center axis M indicated in FIG. 1.

The element 2 can be moved axially out of the element 1 until the end area 19 of the element 2 abuts internally against the guide element 16.

In this way it is possible to ensure a very substantial travel of the element 2 relative to the element 1.

In the present invention it is important that the substantial travel can also be achieved by the fact that the element 2 can be moved out axially in relation to the element 1 with absolute precision under very high forces via the planetary roller system 11, the element 2 being guided via the guide element 16 such that it cannot twist radially relative to the element 1.

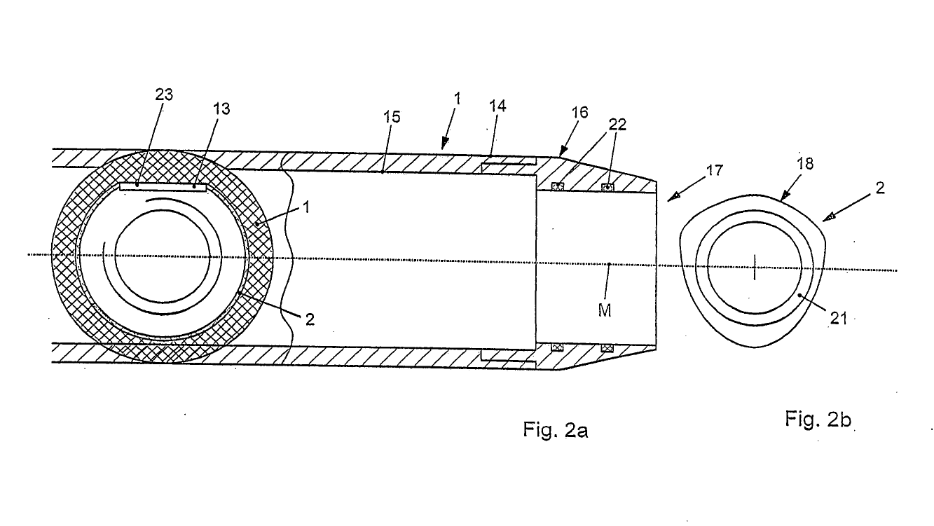

The guide element 16 can have one or more sealing elements 22, as is indicated in FIGS. 1 and 2a. These serve to seal the elements 1 and 2 relative to one another in the end area 19.

However, the scope of the present invention is intended also to cover the case where the element 2 for example is not guided inside the element 1, but instead engages as an outer sleeve over the latter and receives the element 1 inside it and guides it in a manner secure against torsion. In this case, for example, the planet carrier 12 can lie outside the end area 14 of the element 1 and mesh with a corresponding inner thread 21 of the element 2.

The scope of the present invention is also intended to cover the case where, for example, the cross section of the end area 19 of the element 2 has a round, polygonal, many-cornered or other configuration, in order to ensure axial and radial guiding relative to the element 1, in which case a securing against torsion is not absolutely essential, because said securing against radial torsion can be ensured via the guide element 16 between elements 1 and 2.

Between the end area 19 and the guide element 16, a receiving space 23 for accommodating sensors, force sensors, displacement sensors or the like can be provided inside the guide area 15, as is also indicated in FIG. 2a for example.

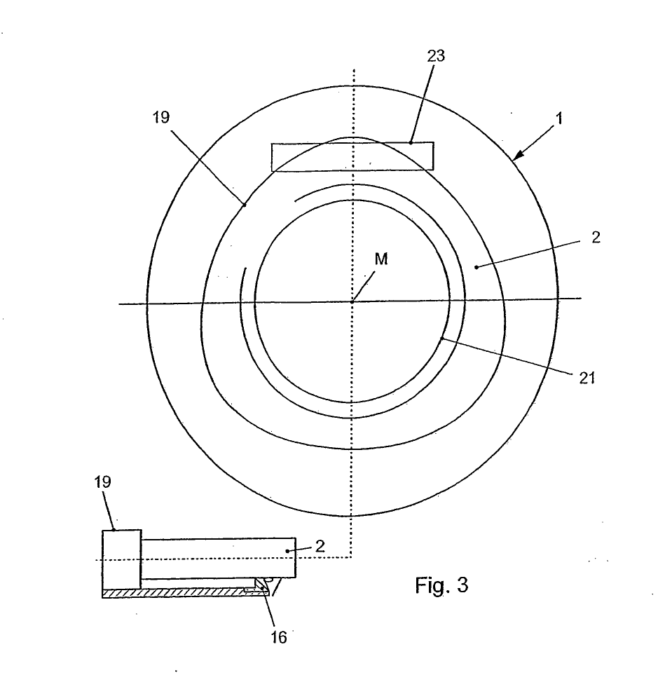

In the illustrative embodiment of the present invention according to FIG. 3, a receiving space 23 is indicated, it also being possible here that only the end area 19 of the element 2 can be configured as a polygonal profile which correspondingly ensures the securing against radial torsion, while the inner guide area 15 of the element 1 likewise has a polygonal configuration. The element 2 adjoining the end area 19 can in this case be configured with a round cross section. Correspondingly, an inner cross section 17 of the guide element 16 of the first element 1 then has a round configuration and is used only for radial and axial guiding. The securing against torsion then takes place only in the end area 19. The invention is not limited to this.

1. A device for extending bones comprising two elements (1, 2) movable relative to one another and interconnected via at least one drive element (7), characterized in that, when the two elements (1, 2) are moved axially in relation to one another, they are guided in a manner secure against relative radial torsion.

2. The device as claimed in claim 1, characterized in that the first element (1) is configured as a receiving sleeve (5) in which at least one radial locking bore (4) is provided at one end.

3. The device as claimed in claim 2, characterized in that the drive element (7) is fitted as electric motor (8) in the receiving sleeve (5) and drives, if appropriate via a drive shaft (10) or directly, a planetary roller system (11) or a thread or spindle system.

4. The device as claimed in claim 3, characterized in that at least one force sensor (13) and/or displacement sensor (13) is assigned to the drive element (7), to the electric motor (8), to the drive shaft (10) and/or to the planetary roller system (11) or spindle system, which sensor (13) is connected for example by spindle systems to the drive element (7), in particular the electric motor (8) with an electronics unit (6).

5. The device as claimed in at least one of claims 1 through 4, characterized in that an inner cross section (17) of the element (1) in the area between electric motor (8) and planetary roller system (11) has a polygonal, rectangular or multi-cornered configuration as a guide area (15) for the second element (2).

6. The device as claimed in claim 5, characterized in that an outer cross section (18) of the element (2) at least partially corresponds to the inner cross section (17) of the first element (1) in the guide area (15) and has a polygonal, rectangular or multi-cornered configuration.

7. The device as claimed in claim 6, characterized in that an inner cross section of the second element (2) is configured as a cylindrical bore which is provided in its inner circumferential surface with a thread (21) which engages with the planetary roller system (11) or thread or spindle system adjacent to the drive shaft (10).

8. The device as claimed in claim 1, characterized in that the second element (2) has at least one radial locking bore (4) at its end.

9. The device as claimed in claim 6, characterized in that, at least at one end, the second element (2) has a polygonal configuration in the area of the outer cross section (18), which engages with an exact fit in the corresponding polygonal inner cross section (17) of the guide area (15) of the first element (1), and, in the end area (14) of the first element (1), a correspondingly configured guide element (16) with polygonal inner cross section (17) ensures that the second element (2) is guided in a manner secure against radial torsion, at least one seal or at least one sealing element (22) being inserted between the elements (1, 2).

10. The device as claimed in claim 9, characterized in that, at one end lying remote from the area (14) of the first element (1), an energy and/or data transmission unit (3), in particular at least one coil for inductive energy and/or data transmission, is inserted which acts in two directions and in a contactless manner and is connected to the drive element (7) and/or the electronics unit (6).

11. The device as claimed in claim 1, characterized in that the element (2) engages as an outer sleeve over the element (1) and receives the element (1) inside it and guides it in a manner secure against torsion.
